(19) United States
(12) Patent Application Publication
(10) Pub. No.: US 2021/0076986 A1
(43) Pub. Date: Mar. 18, 2021

(54) PHYSIOLOGICAL SENSING APPARATUS FOR READING A STRIP

(71) Applicant: BIONIME CORPORATION, Taichung City (TW)

(72) Inventor: Chun-Mu Huang, Taichung City (TW)

(73) Assignee: BIONIME CORPORATION, Taichung City (TW)

(21) Appl. No.: 17/015,382

(22) Filed: Sep. 9, 2020

(30) Foreign Application Priority Data
Sep. 12, 2019 (TW) 108133093

(51) Int. Cl. A61B 5/145; A61B 10/00; G06K 7/02

(52) U.S. Cl. CPC A61B 5/145; A61B 2562/0295; G06K 7/02; A61B 10/0045

(57) ABSTRACT

A physiological sensing device for reading an elongated test strip having an insertion end, a test strip width and a test strip length is provided. The physiological sensing device includes a housing, a test strip slot and a first protruding portion. The housing has a front surface, a rear surface opposite to the front surface, and a lateral surface. The test strip slot is disposed on the lateral surface, and having a first slot wall adjacent to the front surface; a second slot wall adjacent to the rear surface, and disposed opposite to the first slot wall; and a slot opening having a slot width, formed between the first slot wall and the second slot wall for accommodating the elongated test strip, and allowing the insertion end to enter the housing through the slot opening. The first protruding portion extends from the second slot wall to have a protruding width being several times of a width of the slot opening, wherein the test strip length is several times of the test strip width, and the slot width is slightly larger than the test strip width.

### CROSS-REFERENCE TO RELATED APPLICATION AND CLAIM OF

### Priority

The application claims the benefit of Taiwan Patent Application No. 108133093, filed on Sep. 12, 2020, at the Taiwan Intellectual Property Office, the disclosures of which are incorporated herein in its entirety by reference.

### FIELD OF THE INVENTION

The present invention is related to a physiological sensing apparatus, and more particularly to a physiological sensing apparatus for reading a strip.

### BACKGROUND OF THE INVENTION

Patients with chronic diseases, such as diabetes and chronic cardiovascular disease, require daily monitoring of certain physiological parameters, such as changes in blood glucose, blood fat, cholesterol concentration or other parameters, in order to effectively control the condition to avoid deterioration and provide timely treatment. For the needs of home care, handheld physiological sensing devices have emerged on the market one after another.

Common handheld physiological sensing devices are matched with disposable test strips for collecting body fluid samples. When in use, the test strips need to be inserted into the physiological sensing device and reach a certain position. Please refer to FIG. 1, which shows a conventional physiological sensing device 10 used for reading the long physiological test strip 20. The physiological sensing device 10 has a slot 12 on the side of the housing 11 near the middle portion of the side. The slot 12 is exposed on the outer wall 14 of the back of the housing 11 and is visible from the front. When operating the physiological sensing device 10, a user needs to hold one end of the physiological test strip 20 and insert the other end of the physiological test strip 20 toward the slot 12. Since the slot 12 is only slightly wider than the width of the physiological test strip 20, the user should hold the physiological sensing device 10 with one hand and insert the physiological test strip 20 with the other hand. It would be difficult if the operator is an elderly person with poor eyesight or poor arm control ability, because people with poor eyesight or poor arm control cannot easily put the other end of the long physiological test strip 20 into the slot 12, and it is also difficult for the physiological test strip 20 to reach the required reading position after insertion.

Therefore, how to allow the physiological test strip to be easily inserted into the slot of the sensing device for subsequent operations is a technical problem that needs to be solved.

### SUMMARY OF THE INVENTION

The present invention proposes a novel design for a physiological sensing device for reading a long test strip, which allows the user to easily insert the test strip into the physiological sensing device.

In accordance with one aspect of the present invention, a physiological sensing device for reading an elongated test strip having an insertion end, a test strip width and a test strip length is provided. The physiological sensing device includes a housing, a test strip slot and a first protruding portion. The housing has a front surface, a rear surface opposite to the front surface, and a lateral surface. The test strip slot is disposed on the lateral surface, and having a first slot wall adjacent to the front surface; a second slot wall adjacent to the rear surface, and disposed opposite to the first slot wall; and a slot opening having a slot width, formed between the first slot wall and the second slot wall for accommodating the elongated test strip, and allowing the insertion end to enter the housing through the slot opening. The first protruding portion extends from the second slot wall to have a protruding width being several times of a width of the slot opening, wherein the test strip length is several times of the test strip width, and the slot width is slightly larger than the test strip width.

In accordance with another aspect of the present invention, a physiological sensing device for reading a physiological test strip having an insertion end, a test strip width and a test strip length is provided. The physiological sensing device comprises a housing and a test strip slot. The housing has a lateral surface, a front and a rear. The test strip slot is disposed on the lateral surface, and having a first slot wall adjacent to the front; a second slot wall adjacent to the rear; and a slot opening having a slot width, formed between the first and the second slot walls, and configured to allow the insertion end to enter the housing therethrough. The test strip length is several times of the test strip width. The slot width is slightly larger than the test strip width. The second slot wall includes a protruding portion having a protruding width being several times of the test strip width.

In accordance with yet another aspect of the present invention, a physiological sensing device for reading a physiological test strip having an insertion end, a test strip width and a test strip length is provided. The physiological sensing device comprises a housing, a test strip slot and a guiding element. The housing has a lateral surface. The test strip slot is disposed on the lateral surface, and has a first slot wall; a second slot wall being opposite to the first slot wall; and a slot opening formed between the first and the second slot walls, and configured to allow the insertion end to enter the housing therethrough. The guiding element extends from the second slot wall, and has a guiding width being several times of the test strip width.

The objectives and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is a schematic diagram showing a physiological sensing device according to the prior art;

FIG. 2A is a schematic diagram showing a physiological sensing device according to one embodiment of the present invention;

FIG. 2B is a side view of the embodiment of the present invention as shown in FIG. 2A;

FIG. 3A is a schematic diagram showing a physiological sensing device according to another embodiment of the present invention;

FIG. 3B is a side view of the embodiment of the present invention as shown in FIG. 3A;

FIG. 4A is a schematic diagram showing a physiological sensing device according to one another embodiment of the present invention;

FIG. 4B is a side view of the embodiment of the present invention as shown in FIG. 4A;

FIG. 5A is a schematic diagram showing a physiological sensing device according to yet another embodiment of the present invention;

FIG. 5B is a side view of the embodiment of the present invention as shown in FIG. 5A;

FIG. 6 is a schematic diagram showing an operating procedure for the physiological sensing device of the present invention.

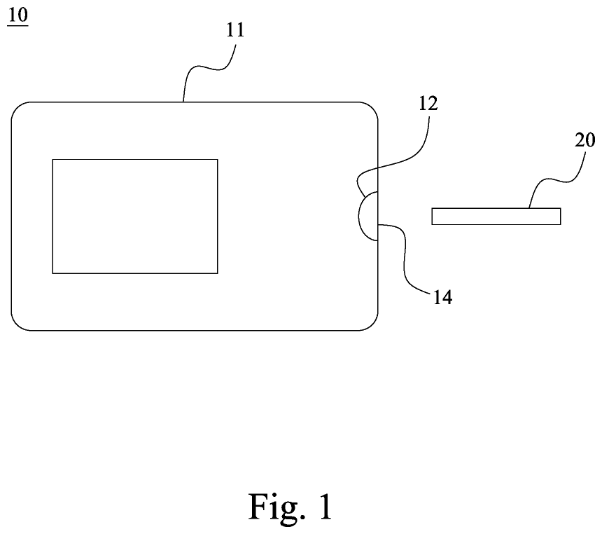

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; they are not intended to be exhaustive or to be limited to the precise form disclosed.

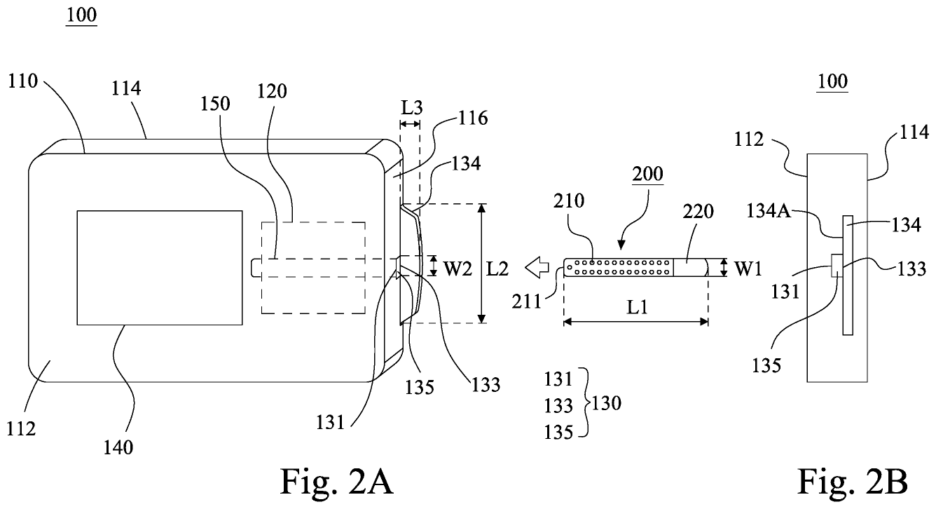

Please refer to FIG. 2A, which shows an embodiment of the physiological sensing device of the present invention. The physiological sensing device 100 has a housing 110 having a front 112, a rear 114 opposite to the front 112, and a lateral surface 116. Based on the operating habits of ordinary people, the front 112 of the housing 110 is often equipped with a display screen 140, which is usually an LCD screen. In other words, if there is no particular rule or limitation, usually the side where the display screen 140 is arranged is regarded as the front side, and the other side relative to the front side is regarded as the back side.

The physiological sensing device 100 is configured to read a physiological test strip 200 whose length L1 is multiple times to the width W1 thereof. Thus, the physiological test strip 200 has an elongated shape. The surface of the coding portion 210 of the physiological test strip 200 is furnished with plural coding elements for example but not limited to in the form of a hole. The coding elements can be, for example, a protrusion, a saw tooth, a row of teeth, a stitch, a slit, a notch, a groove or a through hole. In order to be able to generate a sufficient number of coding combinations, the number of coding elements has to be large, such that the coding portion 210 has a considerable length. The sensing portion 220 of the physiological test strip 200 is provided to carry the specimen or blood sample for the physiological sensing device 100 to read the physiological parameters and perform subsequent analysis. The physiological test strip 200 needs to enter the code-reading position 150 shown by the dashed lines in the physiological sensing device 100, and the physiological parameters can be read after decoding and identification.

As shown in FIGS. 2A and 2B, the physiological sensing device 100 has a test strip slot 130 disposed on the lateral surface 116. The test strip slot 130 has a first slot wall 131 adjacent to the front 112, a second slot wall 133 adjacent to the rear 114, and a slot opening 135 formed between the first slot wall 131 and the second slot wall 133. The function of the slot opening 135 is to allow the insertion end 211 of the physiological test strip 200 (that is, the end close to the encoding portion 210) to pass through and enter the housing 110. In order to restrict the position of the physiological test strip 200 entering the physiological sensing device 100, the slot width W2 of the slot opening 135 only needs to be slightly larger than the test strip width W1, for example, the slot width W2 is between 1.1 and 2 times to the test strip width W1. However, the ordinary people cannot easily insert the physiological test strip 200 into the slot opening 135 if such a design does not have the help of a proper guiding device.

In this embodiment, the physiological sensing device 100 has a protruding portion 134 extending from the position of the second slot wall 133, which has a protrusion width L2 that is multiple times to the width W2 of the slot opening 135. Since the second slot wall 133 is located at the slot opening 135 near the back 114 of the physiological sensing device 100, the user can see the slot opening 135 from the front of the physiological sensing device 100 when plugging the physiological test strip 200 into the test strip slot 130. The protrusion width L2 of the protruding portion 134 is multiple times to the slot width W2, so for the user, when the physiological test strip 200 needs to be inserted into the slot opening 135, the protruding portion 134 can be used as a guide. The user may first let the insertion end 211 of the physiological test strip 200 abut against the guiding surface 134A of the protruding portion 134, and then allow the insertion end 211 to move in a direction along the guiding surface 134A, for example moving downward from the top or moving up from the bottom, and finally inserted into the slot opening 135. The protruding portion 134 has a protrusion length L3, preferably 2 mm or more for example, more preferably 4 mm or even more. The ratio of the protrusion length L3 to the width W1 of the test strip 200 is at least 1:10 or more, more preferably 1:20 or even more, so that the user can easily find the position of the slot opening 135 through the guidance of the protruding portion 134. In one embodiment, the protruding portion 134 can be fixedly disposed on the lateral surface 116 of the housing 110 after being formed. In another embodiment, the protruding portion 134 is connected with the lateral surface 116 of the housing 110 in a detachable manner.

According to an embodiment of the present invention, in order to allow the user to more conveniently insert the physiological test strip 200 to the code-reading position 150 of the physiological sensing device 100, a traction device 120 is disposed in the housing 110 and is adjacent to the slot opening 135. When the insertion end 211 of the physiological test strip 200 enters the housing 110 through the slot opening 135 for a certain distance (not shown), the traction device 120 can be triggered to actively transport the physiological test strip 200 to the code-reading position 150.

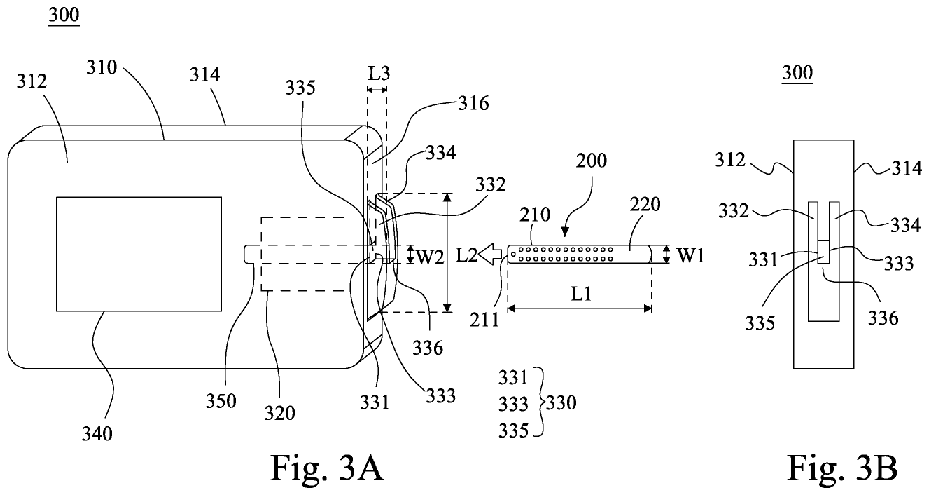

Please refer to FIG. 3A, which shows another embodiment of the physiological sensing device of the present invention. FIG. 3B is a right side view of the embodiment of the present invention as shown in FIG. 3A. The physiological sensing device 300 includes a housing 310 having a front 312, a rear 314, and a lateral surface 316. The front 312 of the housing 310 is configured with a display screen 340. The physiological sensing device 300 also has a test strip slot 330 disposed on the lateral surface 316 of the housing 310. The test strip slot 330 has a first slot wall 331 adjacent to the front 312, a second slot wall 333 adjacent to the rear 314, and a slot opening 335 formed between the first slot wall 331 and the second slot wall 333. The function of the slot opening 335 is to allow the insertion end 211 of the physiological test strip 200 to pass through and enter the housing 310. In order to limit the position where the physiological test strip 200 enters the physiological sensing device 300, the slot width W2 of the slot opening 335 only needs to be slightly larger than the test strip width W1, for example, the slot width W2 is 1.1 to 2 times to the test strip width W1.

In this embodiment, the physiological sensing device 300 not only has a protruding portion 334 extending from the position of the second slot wall 332, but also has another protruding portion 332 extending from the position of the first slot wall 331 and a constraint portion 336 disposed between the protruding portion 334 and the other protruding portion 332 and adjacent to the slot opening 335. The protruding portion 334 and the other protruding portion 332 have a protruding portion width L2 which is multiple times to the slot width W2. Thus, for the user, when the physiological test strip 200 needs to be inserted into the slot opening 335, the protruding portion 334 and the other protruding portion 332 can be used as guiding elements.

The user may first let the insertion end 211 of the physiological test strip 200 enter from the narrow space formed between the protruding portion 334 and the other protruding portion 332, then let the insertion end 211 of the physiological test strip 200 move down between the protruding portion 334 and the other protruding portion 332 and reach the position of the constraint portion 336 under the guidance of the two protruding portions 332, 334. The position of the constraint portion 336 is adjacent to the slot opening 335. The insertion end 211 is now in front of the slot opening 335, so the user can insert the physiological test strip 200 into the slot opening 335 laterally.

The protruding portion 334 has a protruding length L3, preferably 2 mm or longer, more preferably 4 mm or longer, and the ratio between the protruding length L3 and the length L1 for accommodating the test piece is at least 1:20 or more, more preferably 1:10 or more, which allows the users to easily identify and find the position of the slot opening 335. According to an aspect of the present invention, the protruding portion 334, the other protruding portion 332, and the constraint portion 336 can jointly form a guiding member, which can either be fixedly arranged on the lateral surface 316 of the housing 310 after being formed, or be detached for users to install on the lateral surface 316 at a suitable position based on the need.

Likewise, in order to make it more convenient for the user to insert the physiological test strip 200 into the code-reading position 350 of the physiological sensing device 300, a traction device 320 is disposed in the housing 310 of the physiological sensing device 300 and adjacent to the slot opening 335. When the insertion end 211 enters the housing 310 through the slot opening 335 for a certain distance (not shown), the traction device 320 can be triggered to actively transport the physiological test strip 200 to the code-reading position 350.

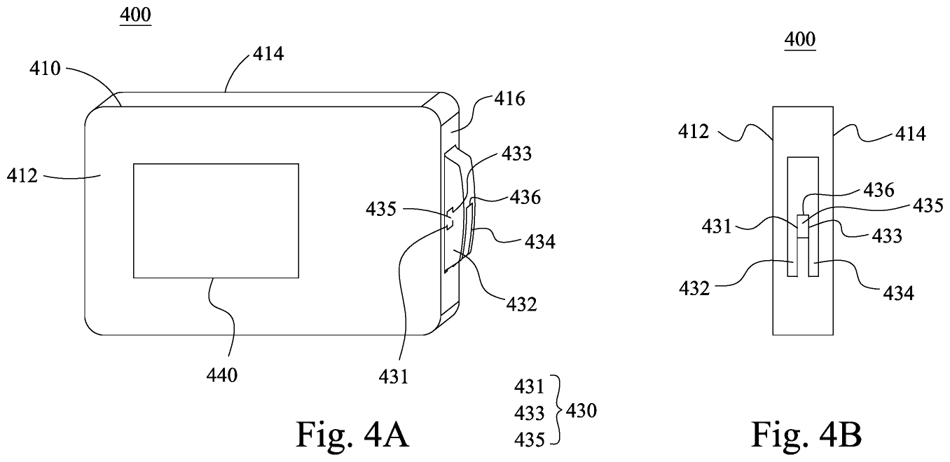

Please refer to FIG. 4A, which shows yet another embodiment of the physiological sensing device of the present invention. FIG. 4B is a right side view of the embodiment of the present invention as shown in FIG. 4A. Similar to the physiological sensing device illustrated in FIG. 3A, the physiological sensing device 400 includes a housing 410 having a front 412, a rear 414, and a lateral surface 416. The front 412 of the housing 410 is configured with a display screen 440. The physiological sensing device 400 also has a test strip slot 430 disposed on the lateral surface 416 of the housing 410. The test strip slot 430 has a first slot wall 431 adjacent to the front 412, a second slot wall 433 adjacent to the rear 414, and a slot opening 435 formed between the first slot wall 431 and the second slot wall 433. The function of the slot opening 435 is to allow the insertion end 211 of the physiological test strip 200 to pass through and enter the housing 410.

In this embodiment, the physiological sensing device 400 has a protruding portion 434 extending from the position of the second slot wall 432, another protruding portion 432 extending from the position of the first slot wall 431 and a constraint portion 436 disposed between the protruding portion 434 and the other protruding portion 432 and adjacent to the slot opening 435. The protruding portion 434 and the other protruding portion 432 have a protruding portion width L2 which is multiple times to the slot width W2. Thus, for the user, when the physiological test strip 200 needs to be inserted into the slot opening 435, the protruding portion 434 and the other protruding part 432 can be used as guiding elements.

The user may first let the insertion end 211 of the physiological test strip 200 enter from the narrow space formed between the protruding portion 434 and the other protruding portion 432, then let the insertion end 211 of the physiological test strip 200 move down between the protruding portion 434 and the other protruding portion 432 and reach the position of the constraint portion 436 under the guidance of the two protruding portions 432, 434. The position of the constraint portion 436 is adjacent to the slot opening 435. The insertion end 211 is now in front of the slot opening 435, so the user can insert the physiological test strip 200 into the slot opening 435 laterally.

The difference between the physiological sensing device 400 shown in FIG. 4A and the physiological sensing device 300 shown in FIG. 3A is that the constraint portion 336 in FIG. 3A is adjacent to bottom of the slot opening 335 while the constraint portion 436 in FIG. 4A is adjacent to the top of the slot opening 435. According to an embodiment of the present invention, the protruding portion 434, the other protruding portion 432, and the constraint portion 436 together constitute a guiding element. When the guiding member is detachably arranged on the housing 410, the devices shown in FIGS. 3A and 4A can be understood as the same guiding element arranged on the side surface 316/416 in different directions. This detachable or rotatable configuration allows either left-handed or right-handed operators to easily operate according to their own convenience.

Similarly, in order to make it more convenient for the user to insert the physiological test strip 200 into the code-reading position 450 of the physiological sensing device 400, a traction device 420 is disposed in the housing 410 of the physiological sensing device 400 and adjacent to the slot opening 435. When the insertion end 211 enters the housing 410 through the slot opening 435 for a certain distance (not shown), the traction device 420 can be triggered to actively transport the physiological test strip 200 to the code-reading position 450.

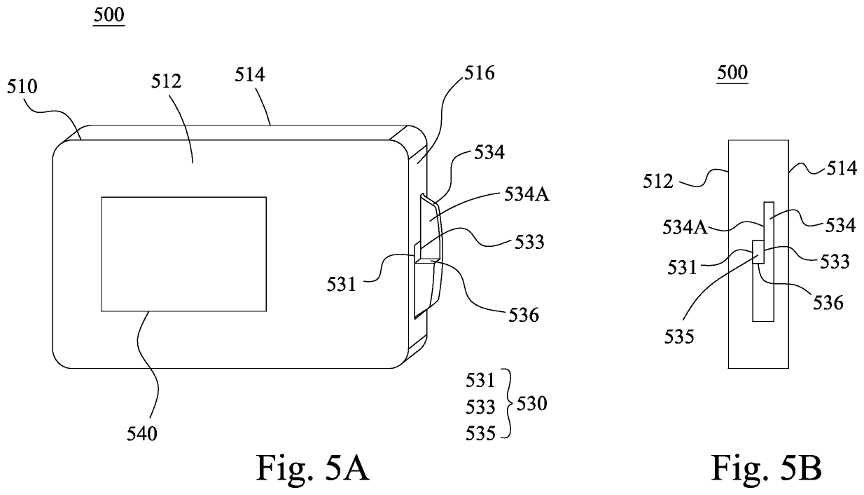

Please refer to FIG. 5A, which shows yet another embodiment of the physiological sensing device of the present invention. FIG. 5B is a right side view of the embodiment of the present invention as shown in FIG. 5A. Similar to the physiological sensing device illustrated in previous sections, the physiological sensing device 500 includes a housing 510 having a front 512, a rear 514, and a lateral surface 516. The front 512 of the housing 510 is configured with a display screen 540. The physiological sensing device 500 also has a test strip slot 530 disposed on the lateral surface 516 of the housing 510. The test strip slot 530 has a first slot wall 531 adjacent to the front 512, a second slot wall 533 adjacent to the rear 514, and a slot opening 535 formed between the first slot wall 531 and the second slot wall 533. The function of the slot opening 535 is to allow the insertion end 211 of the physiological test strip 200 to pass through and enter the housing 510.

In this embodiment, the physiological sensing device 500 has a protruding portion 534 extending from the position of the second slot wall 532 and a constraint portion 536 disposed on the protruding portion 534 and adjacent to the slot opening 535. The protruding portion 534 has a protruding portion width L2 which is multiple times to the slot width W2. Thus, for the user, when the physiological test strip 200 needs to be inserted into the slot opening 535, the protruding portion 534 can be used as a guiding element. The user can let the insertion end 211 of the physiological test strip 200 to be moved downward along the guiding surface 534A of the protruding portion 534 to reach the position of the constraint portion 536. The position of the constraint portion 536 is adjacent to the slot opening 535. The insertion end 211 is now in front of the slot opening 535, so the user can insert the physiological test strip 200 into the slot opening 535 laterally.

According to an embodiment of the present invention, at least part of the test strip slot 130/330/430/530, the protruding portion 134/334/434/534, the other protruding portion 332/432 or the constraint portion 136/336/436/536 can be formed of luminescent material, such as phosphorescent or fluorescent material. When the user needs to operate the physiological sensing device 100/300/400/500 in an environment without sufficient illumination, the test strip slot or the components near the slot can emit light by themselves, providing guidance for the user.

According to another embodiment, at least part of the protruding portion 134/334/434/534, the other protruding portion 332/432, or the constraint portion 136/336/436/536 is made of transparent/translucent material. When the physiological test strip 200 is illuminated by a light source from the slot, it is convenient for the user to perform physiological parameter measurement in the dark. The transparent guiding element allows the user to easily see the position of the test strip slot 130/330/430/530 even in an environment with sufficient light source.

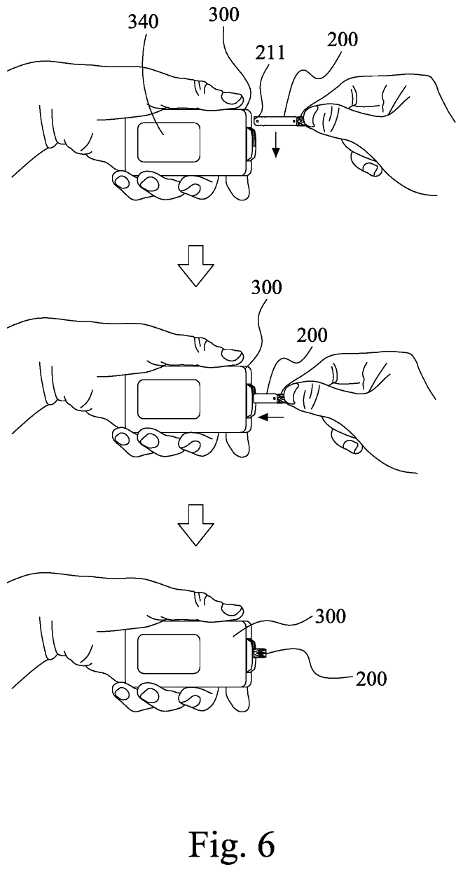

Please refer to FIG. 6, which is a schematic diagram showing an operating procedure for the physiological sensing device of the present invention, taking the embodiment in FIG. 3A as an example. The skilled person in the art can appreciate the operating procedure for the other embodiments of the physiological sensing device.

Please refer to FIG. 3A simultaneously. The upper part of FIG. 6 shows that the user holds the physiological sensing device 300 in one hand and the physiological test strip 200 in the other hand, so that the insertion end 211 is close to the guiding device of the physiological sensing device 300. That is to use the protruding portion 334 and the other protruding portion 332 as a guiding device, first let the insertion end 211 of the physiological test strip 200 enter from the narrow space formed between the protruding portion 334 and the other protruding portion 332.

The middle portion of FIG. 6 shows the user allow the insertion end 211 of the physiological test strip 200 to be moved downward between the protruding portion 334 and the other protruding portion 332 and reach the position of the constraint portion 336. Afterwards, the user can insert the physiological test strip 200 into the slot opening 335 laterally when the insertion end 211 of the physiological test strip 200 have reached the position of the constraint portion 336.

The illustration at the bottom of FIG. 6 shows that the user holds the physiological sensing device 300 furnished with the physiological test strip 200 in hand. After that, the user can perform the steps of blood sampling and physiological parameter analysis as he or she wishes.

Although the operation shown in the figure is that the user holds the physiological sensing device 300 with the left hand and the physiological test strip 200 with the right hand to insert the physiological test strip 200 into the physiological sensing device 300, a left-handed person can also perform the same operation by holding the physiological sensing device 300 with the right hand and the physiological test strip 200 with the left hand.

Due to the guiding device designed in the present invention, the elongated physiological test strip can also be easily inserted into the physiological sensing device by ordinary users, even elderly people without flexible hands, into the slot opening on one side of the sensing device. Obviously, the apparatus and method of the present invention can achieve many effects that cannot be expected by the prior arts.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. A physiological sensing device for reading an elongated test strip having an insertion end, a test strip width and a test strip length, comprising:
a housing having a front surface, a rear surface opposite to the front surface, and a lateral surface;
a test strip slot disposed on the lateral surface, and having:
a first slot wall adjacent to the front surface;
a second slot wall adjacent to the rear surface, and disposed opposite to the first slot wall; and
a slot opening having a slot width, formed between the first slot wall and the second slot wall for accommodating the elongated test strip, and allowing the insertion end to enter the housing through the slot opening; and
a first protruding portion extending from the second slot wall to have a protruding width being several times of a width of the slot opening, wherein the test strip length is several times of the test strip width, and the slot width is slightly larger than the test strip width.

2. The physiological sensing device according to claim 1, wherein at least a portion of the first protruding portion is made of a transparent material or a luminescent material.

3. The physiological sensing device according to claim 1, wherein the first protruding portion is detachably disposed on the lateral surface.

4. The physiological sensing device according to claim 1, further comprising a second protruding portion extending from the first slot wall and opposite to the first protruding portion.

5. The physiological sensing device according to claim 4, wherein the second protruding portion is detachably disposed on the lateral surface.

6. The physiological sensing device according to claim 4, further comprising a constraint portion disposed between the first protruding portion and the second protruding portion and adjacent to the slot opening, wherein the first protruding portion, the second protruding portion and the constraint portion together form a guiding device.

7. The physiological sensing device according to claim 4, wherein at least a portion of the second protruding portion is made of a transparent or a luminescent material.

8. The physiological sensing device according to claim 1, wherein the elongated test strip includes a sensing portion, and a coding portion being adjacent to the insertion end of the elongated test strip.

9. The physiological sensing device according to claim 1, wherein the slot width is 1.1 to 2 times of the test strip width.

10. The physiological sensing device according to claim 1, wherein the first protruding portion has a protruding length being at least one twentieth of the test strip length.

11. The physiological sensing device according to claim 1, further comprising a traction device disposed in the housing, wherein the traction device delivers the elongated test strip to a code-reading position when the insertion end of the elongated test strip enters the housing for a specific depth thereof.

12. A physiological sensing device for reading a physiological test strip having an insertion end, a test strip width and a test strip length, comprising:
a housing having a lateral surface, a front and a rear; and
a test strip slot disposed on the lateral surface, and having:
a first slot wall adjacent to the front;
a second slot wall adjacent to the rear; and
a slot opening having a slot width, formed between the first and the second slot walls, and configured to allow the insertion end to enter the housing therethrough, wherein:
the test strip length is several times of the test strip width;
the slot width is slightly larger than the test strip width; and
the second slot wall includes a protruding portion having a protruding width being several times of the test strip width.

13. The physiological sensing device according to claim 12, wherein at least a portion of the protruding portion is made of a transparent material or a luminescent material.

14. The physiological sensing device according to claim 12, further comprising a constraint portion disposed on the protruding portion and adjacent to the slot.

15. The physiological sensing device according to claim 12, wherein the test strip slot is detachably disposed on the lateral surface.

16. The physiological sensing device according to claim 12, wherein the physiological test strip includes a sensing portion and a coding portion being adjacent to the insertion end of the elongated test strip, and the slot width is 1.1 to 2 times of the test strip width.

17. The physiological sensing device according to claim 12, further comprising a traction device disposed in the housing, wherein the traction device delivers the physiological test strip to a code-reading position when the insertion end of the physiological test strip enters the housing for a specific depth thereof.

18. A physiological sensing device for reading a physiological test strip having an insertion end, a test strip width and a test strip length, and the physiological sensing device comprising:
a housing having a lateral surface;
a test strip slot disposed on the lateral surface, and having:
a first slot wall;
a second slot wall being opposite to the first slot wall; and
a slot opening formed between the first and the second slot walls, and configured to allow the insertion end to enter the housing therethrough; and
a guiding element extending from the second slot wall and having a guiding width being several times of the test strip width.

19. The physiological sensing device according to claim 18, wherein the guiding element is detachably disposed on the second slot wall.

20. The physiological sensing device according to claim 18, wherein at least a portion of the guiding element is made of a transparent material or a luminescent material.
